(19) United States
(12) Patent Application Publication
(10) Pub. No.: US 2026/0216003 A1
(43) Pub. Date: Jul. 30, 2026

(54) AN APPARATUS, KIT AND METHOD FOR MIXING, FILTERING, AND DOSING OF A BIOLOGICAL MATERIAL

(71) Applicant: 1HEALTH GUT INN BALANCE APS, Solrød Strand (DK)

(72) Inventor: Hengameh Chloe LAURIDSEN, Sølrod Strand (DK)

(21) Appl. No.: 19/143,720

(22) PCT Filed: Dec. 21, 2023
(86) PCT No.: PCT/EP2023/087439

§ 371 Date: Jun. 26, 2025

(30) Foreign Application Priority Data
Dec. 28, 2022 (DK) PA202201227

(51) Int. Cl. A61J 3/07; A61B 10/00; A61J 1/10

(52) U.S. Cl. CPC A61J 3/074; A61B 10/0038; A61J 1/10

(57) ABSTRACT

The present invention relates to a kit of a least one polymer bag for collecting, storing, mixing and/or filtering a biological material, the at least one polymer bag comprising a main compartment enclosed by two opposing flat surfaces comprising polymer film, two lateral side walls, a bottom wall, and a top wall, wherein at least one of the two opposing flat surfaces of at least one of the polymer bags of the kit, comprises multiple apertures.

### TECHNICAL FIELD OF THE INVENTION

The present invention relates to a preparation of a biological material, such as a faecal material for preparing a faecal product for gut microbiota transplantation. In particular, the present invention relates to a kit of polymer bags for holding the biological material, a mixing apparatus for mixing, filtering and dosing the faecal material from the kit of polymer bag into a capsule machine for production of capsule for oral intake or oral implant for gut microbiota transplantation, and a method using said kit and apparatus for mixing, filtering and dosing of faecal material.

### BACKGROUND OF THE INVENTION

The human body, and in particular the gastrointestinal tract, contains trillions of bacteria cells, protozoa and viruses, known as the microbiome and a range of metabolites. The microbiome has been linked to many aspects of human health, from gastrointestinal related diseases to obesity.

Focus on faecal microbiota transplantation (FMT) for the prevention and treatment of intestinal disorders has been increasing in the human medicine. Increased clinical applications of FMT have provided convincing proofs that modification of the intestinal microbiota is an effective therapy for intestinal dysbiosis-related diseases.

FMT refers to the process of implanting of a faecal suspension or product obtained from a healthy donor with the right gut microbiota composition into the recipient's intestinal tract to restore the community and function of intestinal microbiota and/or intestinal functionality. FMT, has shown-in randomized, controlled clinical trials-to resolve 80-90% of infections caused by recurrent C. difficile that does not respond to antibiotics.

Faecal microbiota transplants use faeces from a carefully screened healthy donor and transplants the healthy faeces into the colon of the patient. Before the procedure the donor is carefully tested or screened for e.g. communicable infectious diseases according to international guidelines. The donor then gives a stool material which is mixed with a dilutant and blended by shaking or stirring the mixed material and then straining the blended material to separate some of the particulate material. There are multiple routes of administration, and the administration may be rectal, oral or via nasojejunal tube (e.g., via colonoscopy, naso-enteric tube), each of which has unique risks and benefits.

Though the mechanism of gut microbiota transplantation yet has to be precisely determined, it is believed that FMT, in the treatment of C. difficile infection, works by repopulating the patient's microbiome with diverse microorganisms that competitively exclude C. difficile.

In a healthy gut community, C. difficile is outcompeted or outgrown by many different bacterial species. However, receiving antibiotic treatment disrupts this ecosystem by killing those protective bacteria. C. difficile forms spores that are resistant to antibiotics. No longer outcompeted, antibiotic resistant C. difficile may be outgrowing in the gut and produces toxins, which causes severe diarrhoea, abdominal pain, and often fever. With an infusion of FMT from a healthy donor's stool, the C. difficile might be outgrown.

Gut microbiome plays an important role in human and animal health by helping digestion and benefiting the immune system and many other aspects of health. An imbalance of gut microbiota/dysbiosis might be restored by gut microbiota transplantation from a healthy gut, which may contribute to the treatment of various diseases, such as ulcerative colitis and other gastrointestinal conditions.

Methods for preparing the Gut Microbiota Transplantation (FMT) or the faecal microbiota transplantation (FMT) have been continuously developed for improving quality and productivity.

U.S. Pat. No. 4,101,279 (ASLAM MUHAMMED JAVED) discloses a device for collecting and processing stool specimens. A specimen is collected with a bed pan or the like, which specimen is then put in a mixing bowl with a blender to blend the material to obtain a material for analysis. U.S. Pat. No. 3,718,431 (WILD J) discloses a method of stool material collection and testing for the presence of abnormalities such as occult, or hidden blood. A stool specimen is deposited in the device and onto a test medium and then allowed to pass down into the toilet.

US20060122534 (MATSUMURA YASUHIRO et al) discloses a container for filtration of a faecal material for separation of cancer cells from the material. The faecal material is manually pressed through a filter within the container. The complex design of the container makes it expensive, and the document does not disclose how the material is dispersed and a transplant is made after filtering nor how the material is mixed before filtering.

US2021187039 (FELDMAN ARIE et al) discloses a container and a method of preparation of a faecal implant. The container itself contains means for processing the faecal matter into smaller particles and filtering. The complex design of the container makes it expensive, and the manual mixing and processing is tiresome.

WO22015192 (Apartsin Konstantin Anatolevich) discloses a process and apparatus for mixing, filtering and dosing a faecal material into a ready to use faecal implant. The process limits the odour nuisance from the faecal material and manual handling of the material, but the system is complicated and expensive.

Hence, an improved, more efficient, more productive, easier to handle, and less expensive kit of bags and device for handling the fecal material, as well as a more LEAN or straight forward and reproducible method of mixing and filtering said faecal material, especially a method that is less manual and more straight forward throughout the handling process from the fecal material is collected, through the storing, mixing, filtering and dosing into a capsule filling machine for the production of a faecal/gut microbiota capsule implant would be advantageous.

### SUMMARY OF THE INVENTION

Thus, an object of the present invention relates to a kit comprising at least one inexpensive polymer bag for collecting, storing, mixing and/or filtering a biological material and a device for mixing and/or filtering a biological material from the kit of bags.

In particular, it is an object of the present invention to provide a kit comprising at least one inexpensive polymer bag for collecting, storing, mixing and/or filtering a biological material and a device for mixing and/or filtering a biological material from the kit of bags for providing a faecal product for FMT, that solves the above mentioned problems of the prior art with being complex, inefficient, unproductive, complex and requires a lot of manual work and difficult to reproduce.

Thus, an aspect of the present invention relates to a kit of a least one polymer bag for collecting, storing, mixing and/or filtering a biological material, the at least one polymer bag comprising a main compartment enclosed by:
two opposing flat surfaces comprising polymer film,
two lateral side walls,
a bottom wall,
a top wall,
   wherein at least one of the two opposing flat surfaces of at least one of the polymer bags of the kit, comprises multiple apertures.

Yet an aspect of the present invention relates to a kit of a least one polymer bag for collecting, storing, mixing and/or filtering a biological material, comprising a first-bag and a second bag, wherein both the first and the second bag comprise a main compartment enclosed by:
two opposing flat surfaces;
two lateral side walls;
a bottom wall;
a top wall;
   wherein the first and the second bag comprises an inlet on the top wall, wherein the inlet comprising sealing means and fastening means; characterized in that at least one of the two opposing flat surfaces of the polymer first-bag comprises multiple apertures.

A further aspect of the present invention relates to a handling machine for mixing, filtering and dispensing of a biological material, comprising:
i. holding means;
ii. at least one removable and re-attachable mixing paddle;
   characterized in that the machine furthermore comprises a removable and re-attachable movable bar.

Furthermore, one aspect of the present invention relates to a handling machine comprising:
holding means for attaching one or more polymer bag of the kit of the present invention to the holding machine;
one or more mixing paddles for pushing, mixing and filtering the biological material comprised within the one or more polymer bags of the kit of the present invention;
   and movable bar for exerting physical force to the one or more polymer bag of the present invention, squishing the biological material down towards the bottom wall of the polymer bag in a downwards movement.

One aspect of the present invention relates to a method of mixing and/or filtering and/or dispensing of a biological material utilizing the kit of the invention, comprising at least one polymer bag and the holding machine of the invention.

Another aspect of the present invention relates to a method of mixing and/or filtering and/or dispensing of a biological material, utilizing the kit of the present invention comprising at least one polymer bag and the handling machine of the invention, comprising the steps of:
fastening a kit comprising at least one polymer bag to the handling machine, the kit comprising biological material
applying one or more mixing paddles to the kit, or part of the kit, comprising at least one polymer bag

Yet an aspect of the present invention relates to a capsule filling machine for flexible dosing of filtered biological material into capsules and closing/sealing of said filled capsules, said capsule filling machine comprising:
flexible dosing means connected to the dispensing means of the handling machine;
means for holding the polymer bag or hard plastic containing the filtered biological material;
a metal empty capsule separating disc, preferably comprising stainless steel;
a metal empty capsule separating disc, where lower mould, preferably comprising stainless steel, said lower mould further comprising multiple holes and spit-ejector pins.
a metal empty capsule filling/dosing disc, comprising multiple holes and spit-ejector pins.

A further aspect of the present invention relates to a capsule filling machine for flexible dosing of filtered biological material into capsules, closing/sealing, freezing and disposing of said filled capsules, said capsule filling machine comprising:
flexible dosing means connected to the dispensing means of the handling machine;
means for holding the polymer bag or hard plastic containing the filtered biological material;
a metal empty capsule separating disc, preferably comprising stainless steel;
a metal empty capsule separating disc, where lower mould, preferably comprising stainless steel, said lower mould further comprising multiple holes and spit-ejector pins.
a metal empty capsule filling/dosing disc, comprising multiple holes and spit-ejector pins.

An even further embodiment of the present invention relates to a method of mixing and/or filtering and/or dispensing of a biological material, utilizing the kit according to the present invention and the handling machine of the present invention, comprising the step of:
fastening a kit comprising at least one polymer bag to the handling machine, the kit comprising biological material,
   applying one or more mixing paddles to the kit, or parts of the kit, attached to the handling machine.

Yet an aspect of the present invention relates to a method of preparing a faecal/gut microbiota capsule implant, the method comprising the steps of:
providing a faecal composition, preferably by the invention according to the present invention; and
filling the faecal composition into at least a first coating, providing a first coated faecal composition.

A further embodiment of the present invention relates to a faecal capsule implant obtainable by the present invention.

A further embodiment of the present invention relates to a faecal capsule implant comprising a single acid sensitive hard capsule.

Yet another aspect of the present invention relates to a faecal capsule implant (or a gut microbiota capsule implant) according to the present invention for use as a medicament.

### BRIEF DESCRIPTION OF THE FIGURES

FIG 1 shows the polymer first-bag of the kit of the present invention.

FIG. 2 shows the polymer first-bag of the kit of the present invention, with attached separate sheet of polymer attached to one of the two opposing flat sides of the bag.

FIG. 3 shows the polymer first-bag of the kit of the present invention, with thin polymer film attached to at least one of the two opposing flat sides of the bag.

FIG. 4 shows different cross-sections of the polymer first-bag of the kit of the present invention.

FIG. 5 shows the polymer second bag of the kit of the present invention.

FIG. 6 shows an embodiment of the present invention where separate sheet of polymer comprises two layers, that is the polymer third-bag.

FIG. 7 shows the polymer bag design.

FIG. 8 shows another polymer bag design.

FIG. 9 shows the handling machine of the present invention, with two mixing paddles attached.

FIG. 10 shows the handling machine of the present invention, with a movable bar attached.

FIG. 11 shows the handling machine of the present invention, with two mixing paddles attached.

FIG. 12 shows the handling machine of the present invention, with a movable bar attached.

FIG. 13 shows the process of the liquid capsule filling machine according to the present invention.

FIG. 14 shows a capsule holder used in the liquid capsule filling machine according to the present invention.

The present invention will now be described in more detail in the following.

### DETAILED DESCRIPTION OF THE INVENTION

The inventor of the present invention surprisingly found a new kit comprising at least one polymer bag showed highly effective, easy to handle when collecting, storing, mixing and/or filtering a biological material, in particular faecal material when providing faecal capsule implants, and allowing a more straight forward and reproducible method and which is gentler to the microorganisms present.

Thus, one embodiment of the present invention relates to a kit comprising at least one inexpensive polymer bag for collecting, storing, mixing and/or filtering a biological material in a simple streamlined manner. Another embodiment of the present invention is a handling apparatus that utilizes the specific design of the kit of the present invention, for handling, e.g. mixing, filtering and/or dosing a biological material comprised within the kit. Yet another embodiment of the present invention is a method of sampling, storing, mixing, filtering and/or dosing of a biological material utilizing the kit of polymer bags and the specialized handling apparatus of the invention.

Thus, one embodiment of the present invention relates to a kit of polymer bags for collecting, storing, mixing, and/or filtering of a biological material, comprising at least one polymer bag.

The kit of the present invention may comprise at least one polymer bag or polymer container or polymer pouch. In the following the term “polymer bag” may be used for simplicity, however, the at least one polymer bag may be substituted by the polymer container or polymer pouch. The kit of the present invention may preferably comprise at least one polymer bag.

In an embodiment of the present invention, the polymer bag may be a single layered polymer bag or a multi-layered polymer bag.

The bag according to the present invention may be made from a polymer material (a polymer bag) or a metal material (a metal bag). In an embodiment of the present the terms “bag” or “polymer bag” may be used interchangeably, and the skilled person would know that the terms “bags” and “polymer bags” may relate to bags made from a plastic material, metal material, or a combination hereof, however, a plastic material may be preferred.

In an embodiment of the present invention at least one of the polymer bags, comprises a polymer film. One embodiment of the present invention provides a kit comprising at least one polymer bag and a separate polymer film, wherein any bag or film comprises a polymer film.

The use of polymer film for production of different objects, such as different containers is well known. By creating a film comprising different layers of polymer distinct characteristics and functionality of the film can be achieved, such as a film with an outer layer and/or layers that protects against environmental impacts while the inner layer and/or layers can have such characteristic as providing protection of the content of the container and/or permeability suitable for the content of the container.

One embodiment of the present invention relates to a kit comprising at least one polymer bag or separate sheet of polymer comprising:
solid polymer film
the solid polymer film comprising at least one layer comprising odour-barrier
   wherein the odour-barrier comprises Polyvinylidene Chloride (PVDC) and/or Ethylene Vinyl Alcohol (EVOH) and/or PolyVinyl Alcohol (PVOH) and/or polyamide homo-, co-, or ter-polymers, such as a core layer of PVDC and/or oriented polypropylene (OPP) and/or Polyethylene (PE) terephthalate (PET) and/or cast polypropylene (CPP).

One embodiment of the present invention relates to a kit comprising at least one polymer bag or separate sheet of polymer comprising:
solid polymer film
the solid polymer film comprising an outer layer comprising flexible Polyvinyl PVC, EVOH, PVOH, PVDC, OPP, PE, PET, CPP.

One embodiment of the present invention relates to a kit comprising at least one polymer bag or separate sheet of polymer comprising:
solid polymer film
the solid polymer film comprising at least one layer comprising odour-barrier
the solid polymer film comprising an outer layer comprising flexible Polyvinyl, oriented polypropylene (OPP) and/or Polyethylene terephthalate (PET)and/or cast polypropylene (CPP)
   wherein the flexible PVC is highly permeable to oxygen and/or carbon dioxide and the odour-barrier comprises Polyvinylidene and/or Ethylene Vinyl Alcohol (EVOH) and/or PolyVinyl Alcohol (PVOH) and/or polyamide homo-, co-, or ter-polymers, such as a core layer of PVDC, oriented polypropylene (OPP) and/or Polyethylene (PE) terephthalate (PET) and/or cast polypropylene (CPP)

Another embodiment of the present invention relates to a kit comprising at least one polymer bag. In one embodiment of the invention the kit comprises polymer first bag and a polymer second bag. In another embodiment of the invention the kit of polymer bags comprises a separate sheet of polymer. In yet another embodiment the separate sheet of polymer is formed as a bag and is referred to as a polymer third bag.

One embodiment of the present invention relates to a kit comprising at least one polymer bag comprising a main compartment enclosed by:
opposing flat surfaces comprising two sheets of polymer film
two lateral side walls
a bottom wall
top wall

There are different methods of closing or attaching the two opposing sheets of polymer film for constructing the walls e.g. multiple types of welding, gluing by different adhesives or solvents, by heat-sealing, brazing or soldering.

One embodiment of the present invention relates to a kit comprising at least one polymer bag comprising fastening means, for attaching the bag and/or bags to a handling machine. The fastening means may be constructed to be attached to suitable fastening means on a handling machine, e.g. a handling machine according to the present invention, for keeping the bags attached to the machine during mixing and filtering and/or for attaching the bag to the capsule filling machine, e.g. during dosing of biological material into capsules. There are many different methods known in the art for attaching a polymer bag and/or polymer bags to an object, e.g. such as clipping the bag and/or bags to the object or providing the bag and/or bags with holes for hanging the bag and/or bags on a protruding part of the object.

One embodiment of the present invention relates to a kit comprising at least one polymer bag comprising a main compartment enclosed by:
opposing flat surfaces comprising two sheets of polymer film
two lateral side walls
a bottom wall
top wall
fastening means
   wherein the fastening means are suitable for being attached to a clip or hanging the bag and/or bags to a protrusion.

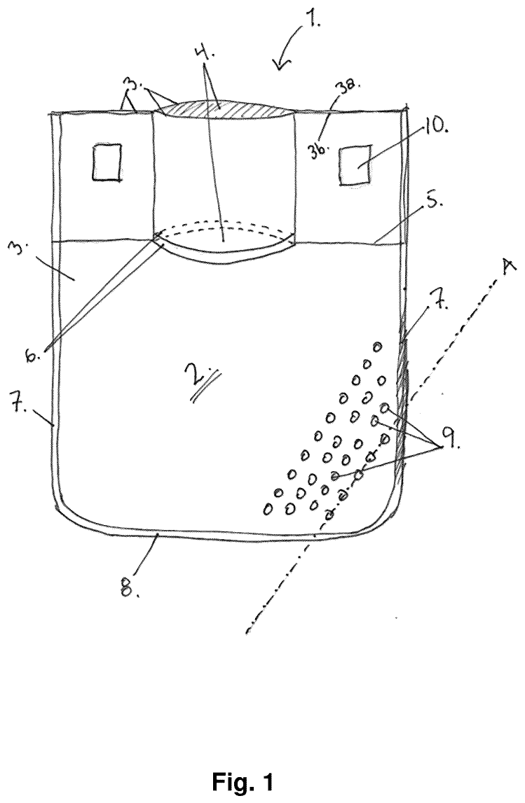

E.g. in reference to FIG. 1, one embodiment of the present invention, provides a first bag (1) comprising a main compartment (2) enclosed by two opposing flat sides (3a and 3b), two side walls (7), a bottom wall (8), fastening means (10), a top wall (5) with an inlet (4), the inlet comprising sealing means (6) enabling air-tight sealing of the inlet (4).

There are multiple known methods in the art on how to design an inlet on a polymer container that can be opened and air-tightly closed, such as polymer zipper as commonly used for storing food products or a strip and/or strips of suitable adhesive that closes the inlet when pressure is applied.

One embodiment of the present invention relates to a kit comprising a polymer bag and/or bags wherein the sealable means (6 or 26) of the inlet (4 or 24) comprise a polymer zipper. Another embodiment of the present invention relates to a kit comprising a polymer bag and/or bags wherein the sealable means (6 or 26) of the inlet (4 or 24) comprise adhesive strip and/or strips.

One embodiment of the present invention relates to a kit comprising a first-bag (1) comprising a main compartment (2) enclosed by:
opposing flat surfaces comprising two sheets of polymer film (3a and 3b)
two lateral side walls (7)
a bottom wall (8)
a top wall (5)
   wherein the top wall comprises an inlet (4) constructing an opening into the main compartment (2) and wherein the inlet comprises sealing means (6), enabling manual and/or mechanical airtight closing of the inlet and wherein the inlet (4) constructs an opening into the main compartment for receiving the biological material.

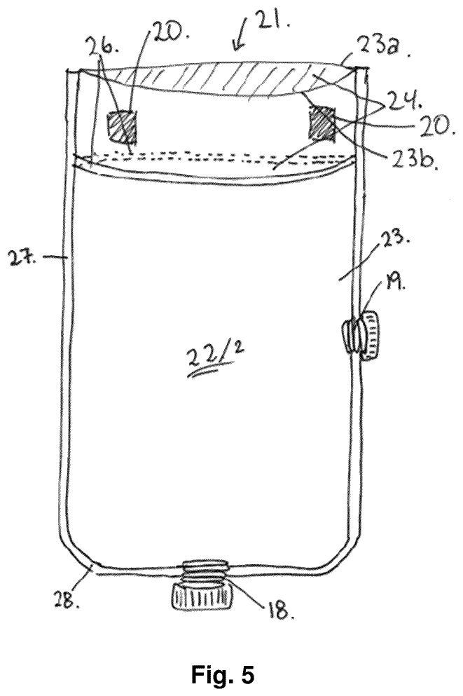

E.g. in reference to FIG. 5, one embodiment of the present invention provides a second-bag (21) comprising a main compartment (22) enclosed by two opposing flat sides (23a and 23b), two lateral side walls (27), a bottom wall (28), sealing means constructing a top wall (26) and when the sealing means are open, constructing an inlet (24), fastening means (20), vacuum inlet (19) and dosing outlet (18), wherein the sealable inlet (24) is constructed for receiving the first-bag of the kit of the present invention.

Another embodiment of the present invention relates to a kit comprising a second-bag comprising a separate top wall, not shown in FIG. 5, wherein the top wall of the second-bag furthermore comprises a sealable inlet.

One embodiment of the present invention relates to a kit comprising a second-bag (21) comprising a main compartment (22) enclosed by:
opposing flat surfaces (23a and 23b) comprising two sheets of polymer film
two lateral side walls (27)
a bottom wall (28)
fastening means (20)
a top wall either constructed by sealable means (26) containing an inlet (24); or a top wall comprising an inlet (24) comprising sealable means (26)
   wherein the inlet (24) constructs an opening into the main compartment (22) for receiving first-bag (1) of the present invention.

One embodiment of the present invention relates to a kit comprising a second-bag (21) comprising a main compartment (22) enclosed by:
opposing flat surfaces (23a and 23b) comprising two sheets of polymer film
two lateral side walls (27),
one of the two lateral side walls (27) comprising a closed vacuum inlet (19)
a bottom wall (28)
the bottom wall comprising a closed dosing outlet (18)
fastening means (20)
a top wall either constructed by sealable means (26) containing an inlet (24); or a top wall comprising an inlet (24) comprising sealable means (26)
   wherein the inlet (24) constructs an opening into the main compartment (22) for receiving first-bag (1) of the present invention, and wherein both the dosing outlet (18) is opened by attaching it to a dispensing means and the vacuum inlet (19) is opened by attaching it to vacuum means.

Vacuum is commonly utilized on polymer bags, e.g. for packaging different kind of both consumable such as food or medicine or for other purposes. A common attachment means for applying vacuum on a polymer bag is to screw vacuum means, e.g. vacuum pipe, vacuum tube to the polymer bag.

One embodiment of the present invention relates to a kit wherein the vacuum inlet (19) is opened by screwing it to vacuum means, e.g. vacuum pipe, vacuum tube.

A preferred embodiment of the present invention relates to a capsule filling machine (such as a dosing liquid capsule filling machine) for providing a faecal capsule implant according to the present invention. The capsule filling machine may comprise means for holding the faecal composition in fluid contact to a flexible dosing means.

One embodiment of the present invention relates to a kit wherein said flexible dosing means may be e.g. a dosing pipe; a dosing disc or dosing arm of a dosing capsule filling machine, as known in the art.

One embodiment of the present invention relates to a kit comprising a second-bag (21) comprising a main compartment (22) enclosed by:
opposing flat surfaces (23a, 23b) comprising two sheets of polymer film
two lateral side walls (27)
a top wall (26)
a bottom wall (28) comprising dosing outlet (18)
   wherein the dosing outlet is opened by attaching a flexible dosing means at the dosing outlet, thereby constructing a fluid contact the main compartment of the second-bag and a dosing disc of a capsule filling machine (e.g. a dosing liquid capsule filling machine), and/or dosing arm of a dosing capsule machine and/or a dosing pipe connected to a dosing capsule filling machine.

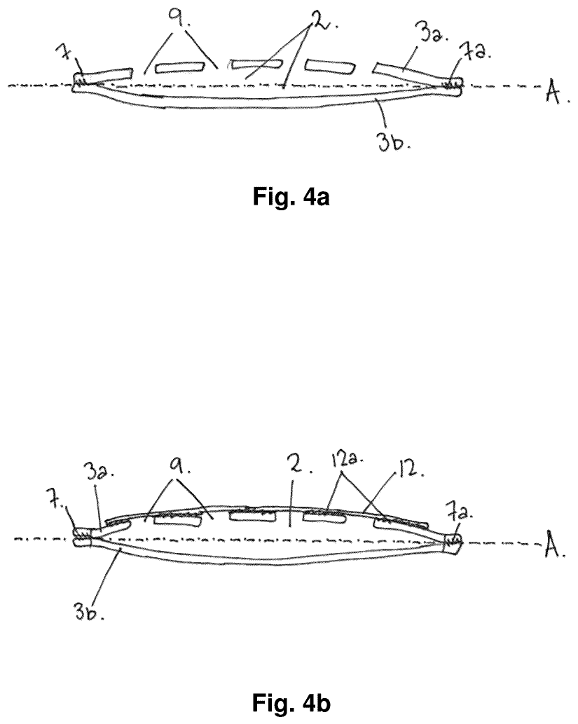

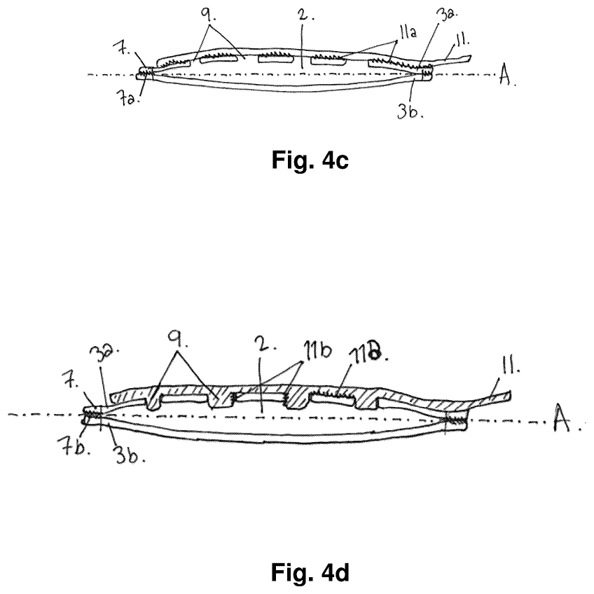

E.g. in reference to FIG. 1 and FIG. 4a, one embodiment of the present invention, provides a first bag (1) comprising a main compartment (2) enclosed by two opposing flat sides (3a and 3b), wherein at least one of the two opposing flat sides comprises multiple apertures (9), the multiple apertures constructing multiple openings or filtering means for filtering the parts of the biological material that has smaller particle size than the diameter of the multiple apertures, from the main compartment into the bags environment.

One embodiment of the present invention relates to a kit comprising a first bag, comprising a main compartment enclosed by:
two opposing flat surfaces (3a, 3b) comprising two sheets of polymer film, at least one of the two opposing surfaces comprising multiple apertures (9)
two lateral side walls (7)
a top wall (5)
a bottom wall (8)
   wherein the multiple apertures may have a diameter in the range of 0.1-3.5 mm, e.g. in the range of 0.3-3.0 mm, such as in the range of 0.5-2.5 mm, e.g. in the range of 0.7-2.0 mm, such as in the range of 1.0-1.75 mm, in the range of 1.2-1.5 mm. For some applications the multiple apertures may have a diameter between 0.1 and 0.9 mm, such as between 0.2 and 0.7 mm, e.g. between 0.2 and 0.5 mm, such as between 0.3 and 0.4 mm, e.g. between 0.35 and 0.44 mm.

In an embodiment of the present invention the only one of the two opposing surfaces (3a or 3b) comprise multiple apertures (9). In another embodiment of the present invention both of the two opposing surfaces (3a and 3b) comprise multiple apertures (9).

The multiple apertures according to the present invention may be provided as holes, e.g. welded in at least part of the bag or as a net inserted into at least part of the bag, preferably in the polymer first bag. When the net or the holes are not covered e.g. by a polymer sheet, the net or the holes provide an opening from the inside of the bag in which it is introduced to the outside of the bag.

The net may be provided in a polymer material, a plast material, a fibre material, a metal material, or the like.

In an embodiment of the present invention the multiple apertures (or the holes, or openings of the net) may have a diameter in the range of 0.1-3.5 mm, e.g. in the range of 0.3-3.0 mm, such as in the range of 0.5-2.5 mm, e.g. in the range of 0.7-2.0 mm, such as in the range of 1.0-1.75 mm, in the range of 1.2-1.5 mm. For some applications the multiple apertures may have a diameter in the range of 0.1-0.9 mm, preferably a diameter in the range of 0.2-0.5 mm, more preferably in the range of 0.3-0.4 mm, even more preferably a diameter around 0.4 mm.

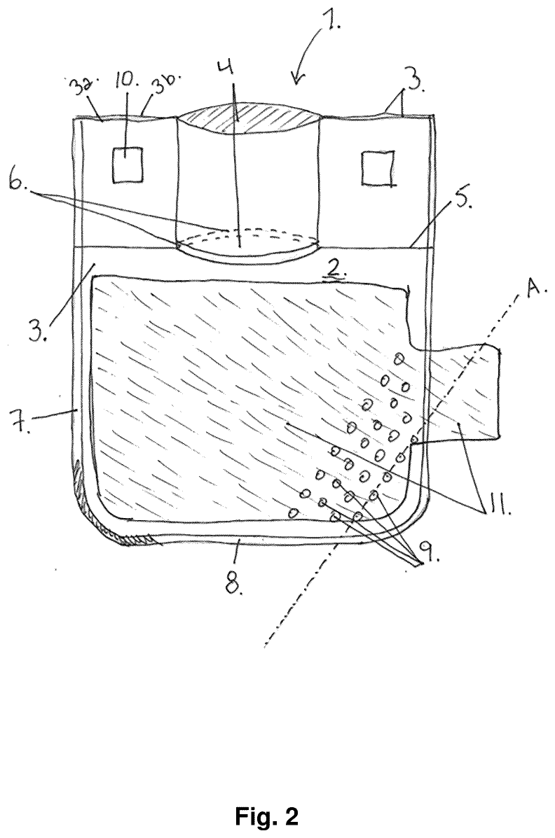

E.g. in reference to FIGS. 1, 2 and 4, one embodiment of the present invention provides a kit comprising a first-bag (1) wherein the multiple apertures (9) may be closed by a separate polymer sheet (11) and wherein the separate polymer sheet (11) can be removed from the first bag (1) by either manual or mechanical pulling.

The multiple apertures may initially be closed by a thin film (e.g. a polymer sheet), and the multiple apertures may be activated or opened by removing the thin film and filtering the parts of the biological material that has smaller particle size than the diameter of the multiple apertures may be allowed to sieve through the multiple apertures providing a separation of biological material.

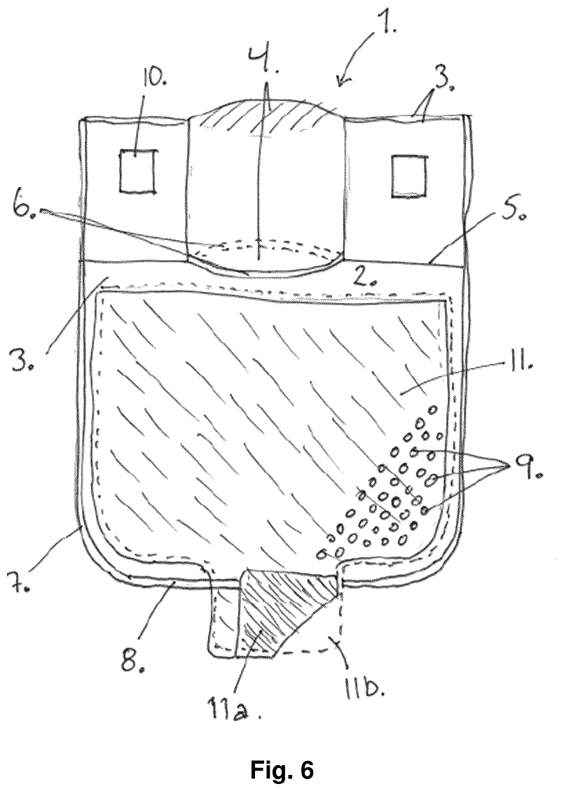

E.g. in reference to FIGS. 1, 2, 4 and 6, it should be noted that the separate polymer sheet (11) can have different form, shape, size, and placement.

One embodiment of the present invention provides a kit comprising a first-bag (1) wherein the multiple apertures (9) are closed by two separate polymer sheets (11) that can be removed from the first bag (1) by either manual or mechanical pulling and wherein means for pulling are placed at the bottom of the kit.

Another embodiment of the present invention provides a kit comprising a first-bag (1) wherein the multiple apertures (9) are closed by a third bag (50) and wherein the third-bag (50) can be removed from the first bag (1) by either manual or mechanical pulling.

One embodiment of the present invention relates to a kit comprising a first-bag (1) comprising a main compartment (2) enclosed by two opposing flat sides (3a and 3b), wherein at least one of the two opposing sides (3a, 3b) comprises:
multiple apertures (9)
the multiple apertures may be closed by a separate polymer sheet (11)
the separate polymer sheet (11) comprising protrusions or buttons for closing the apertures wherein flat side (3a) comprising the apertures (9) and the separate polymer sheet (11) may be attached/bonded either on the of the opposing flat side (3a) by bond (11a) or attached/bonded by the protrusions or buttons of sheet (11) and the apertures (9) by bond (11b) and wherein the separate polymer sheet (11) can be removed from the first bag (1) by either manual or mechanical pulling.

One embodiment of the present invention relates to a kit comprising a first-bag (1) comprising a main compartment (2) enclosed by two opposing flat sides (3a and 3b), wherein at least one of the two opposing sides (3a, 3b) comprises:
multiple apertures (9)
the multiple apertures may be closed by a separate polymer sheet (11)
   wherein the sheet (11) may be attached/bonded to the surface of the flat side (3a) by bond (11b) and wherein the separate polymer sheet (11) can be removed from the first bag (1) by either manual or mechanical pulling.

One embodiment of the present invention relates to a kit comprising a first-bag (1) comprising a main compartment (2) enclosed by two opposing flat sides (3a and 3b), wherein at least one of the two opposing sides (3a, 3b) comprises:
multiple apertures (9)
the multiple apertures may be closed by a third-bag (50)
the third-bag (50) comprising protrusions or buttons for closing the apertures
   wherein flat side (3a) comprising the apertures (9) and the third-bag (50) may be attached/bonded either on the of the opposing flat side (3a) by bond (11a) or attached/bonded by the protrusions or buttons of third-bag (50) and the apertures (9) by bond (11b) and wherein the third-bag (50) can be removed from the first bag (1) by either manual or mechanical pulling.

One embodiment of the present invention relates to a kit comprising a first-bag (1) comprising a main compartment (2) enclosed by two opposing flat sides (3a and 3b), wherein at least one of the two opposing sides (3a, 3b) comprises:
multiple apertures (9)
the multiple apertures may be closed by a third-bag (50)
   wherein the third-bag (50) may be attached/bonded to the surface of the flat side (3a) by bond (11b) and wherein the third-bag (50) can be removed from the first bag (1) by either manual or mechanical pulling.

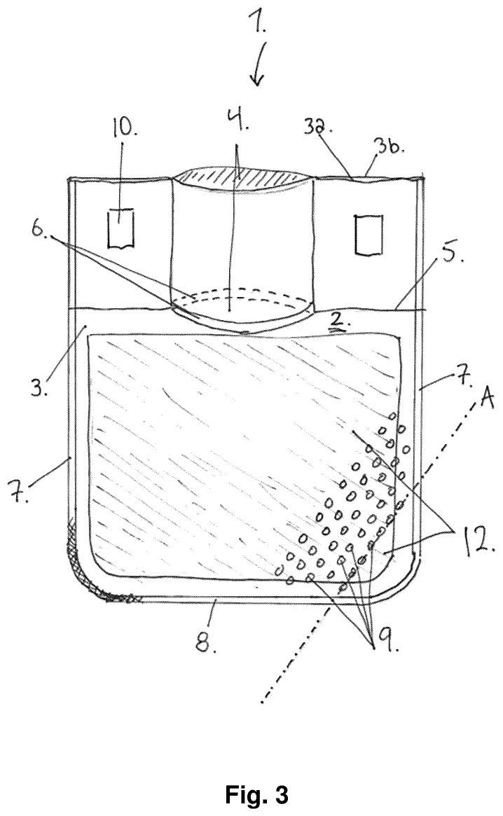

E.g. in reference to FIGS. 1, 3 and 4, one embodiment of the present invention provides a kit comprising a first-bag (1) wherein the multiple apertures (9) are closed by thin polymer film (12) and wherein the thin polymer film (12) can be ruptured by physical force.

One embodiment of the present invention relates to a kit comprising a first-bag (1) comprising a main compartment (2) enclosed by two opposing flat sides (3a and 3b), wherein at least one of the two opposing sides (3a, 3b) comprises:
multiple apertures (9)
the multiple apertures may be closed by a thin polymer film (12)
   wherein the thin polymer film (12) may be attached/bonded to the surface of the flat side (3a) by bond (12b) and wherein the thin polymer film (12) can be ruptured by physical force thereby opening the multiple apertures (9).

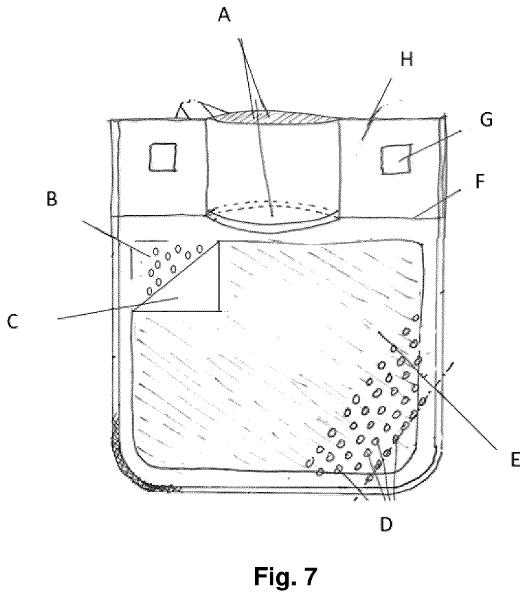

E.g. in reference to FIG. 7, one embodiment of the present invention provides a kit comprising a first-bag wherein the multiple apertures (D) are closed by a separate polymer sheet (C) and wherein the separate polymer sheet (C) can be wherein the sheet is attached/bonded/sealed/ glued to the surface of the flat side (B) and wherein the separate polymer sheet (C) can be removed from the first bag by either manual or mechanical pulling.

In an embodiment of the present invention, a first bag as shown in FIG. 7 may be provided, with the similar description as described in respect of FIG. 1, where the bag 1 may be enclosed by two opposing flat sides H, two side walls (E, C), a bottom wall, fastening means (G), a top wall with an inlet (A), the inlet comprising sealing means (A) enabling air-tight sealing of the inlet (F).

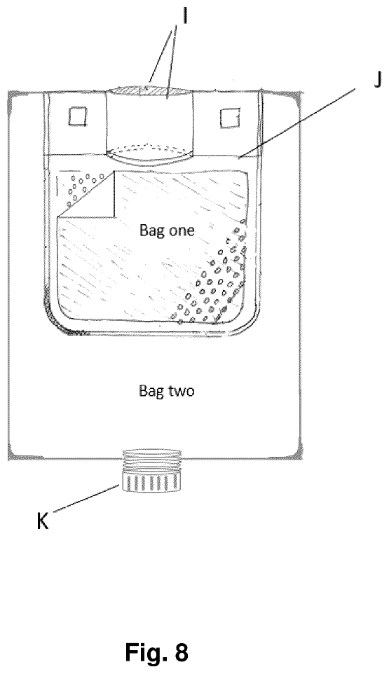

E.g. in reference to FIG. 8, another design of the present invention may be presented. The design presented in FIG. 8 provides a second bag just as described in the FIG. 5, with one dosing outlet (K). The second bag fastening, glued and or sealed with bag one comprising a main compartment enclosed by two opposing flat sides, two lateral side walls, a bottom wall, sealing means constructing a top wall and when the sealing means may be open, constructing an inlet, fastening means, and dosing outlet (K), wherein the sealable inlet may be constructed for receiving the first-bag of the kit of the present invention as described in respect of FIG. 5.

Another embodiment of the present invention relates to a kit comprising a second bag comprising a top wall, wherein the top wall of the second-bag seal and or glued to inlet to first bag. The second bag may be bigger than bag 1, enabling filtering and/or pressing the product from bag 1 into bag 2 for dosing.

The first bag may be removed from the second bag. Preferably the first bag may be removed from the second bag before the filtered biological material, e.g. the filtered faecal composition, may be provided to the capsule filing machine.

In an embodiment of the present invention the kit may be provided as a single bag comprising at least 2 compartments, such as at least 3 compartments. Preferably, the first compartment may be placed adjacent to the second compartment, the third compartment may be placed adjacent to the second compartment and/or the second compartment may be placed between the first and third compartments.

The first compartment may be provided for mixing the product (as described herein), the second compartment may be provided for filtering the product (as described herein), and the third compartment may be provided for dosing of the filtered product (as described herein).

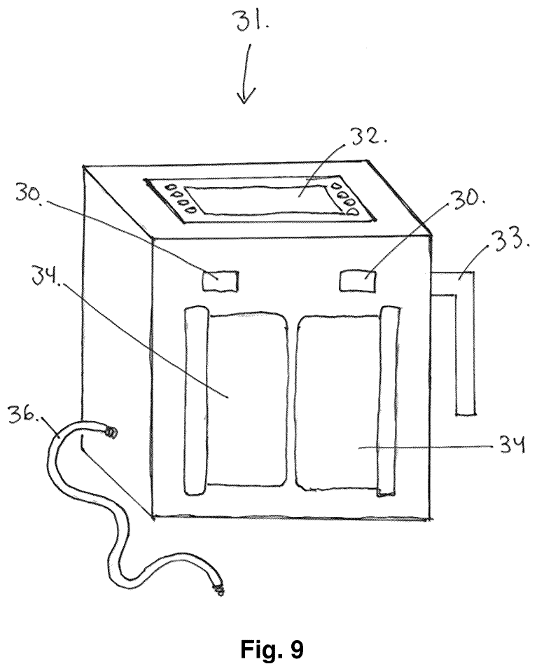

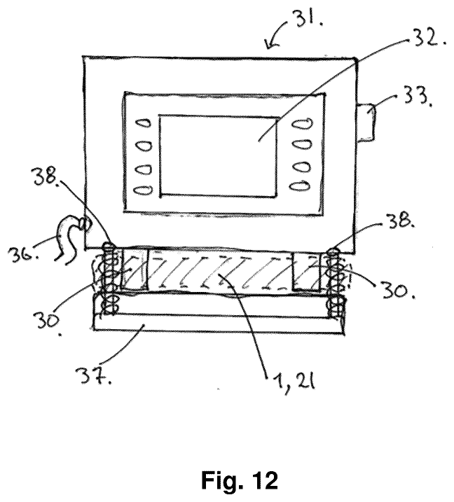

E.g. in reference to FIGS. 9, 10, 11 and 12, one embodiment of the present invention may provide a handling machine (31) comprising a control display (32), handle (33) and holding means (30), the handling machine constructed for mixing and/or filtering and/or dosing of the biological sample comprised within the kit of the present invention.

One embodiment of the present invention provides a handling machine (31) comprising holding means (30) for holding and/or attaching one or more polymer bags of the kit of the present invention to the handling machine, the holding means (30) comprising one or more clip or one or more protrusion.

Different types of holding and/or attachment means are known in the art, for attaching a polymer bag to an object, e.g. the handling machine. Such attachments means include, but are not limited to a clip, clamp, pin or other kind of a protrusion.

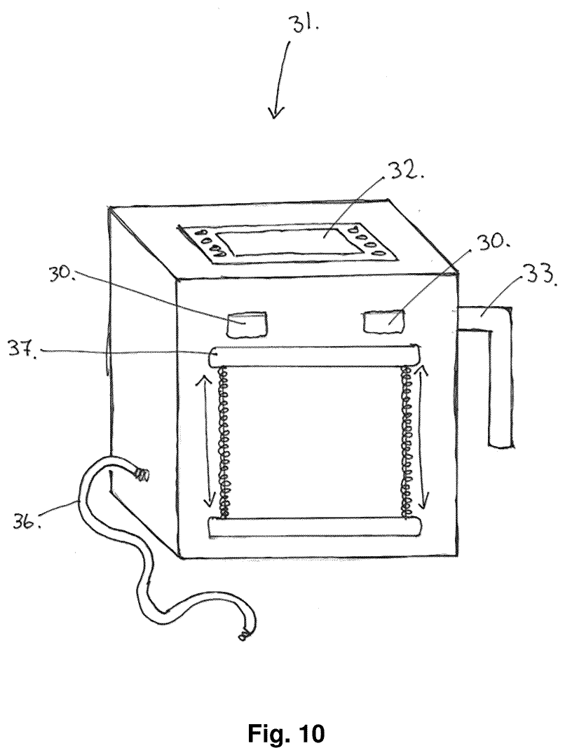

In reference to FIGS. 9 and 10, one embodiment of the present invention provides a handling machine (31), the handling machine comprising:
holding means (30),
one or more mixing paddles (34),
   wherein the mixing paddles are constructed for exerting physical force to the surface of the polymer bag and/or bags attached to the holding means (30) of the handling machine (31).

Preferably, the physical force applied by the mixing paddles swinging back-and-forth and thereby hitting/pushing the surface of the polymer bag and/or bags attached to the machine.

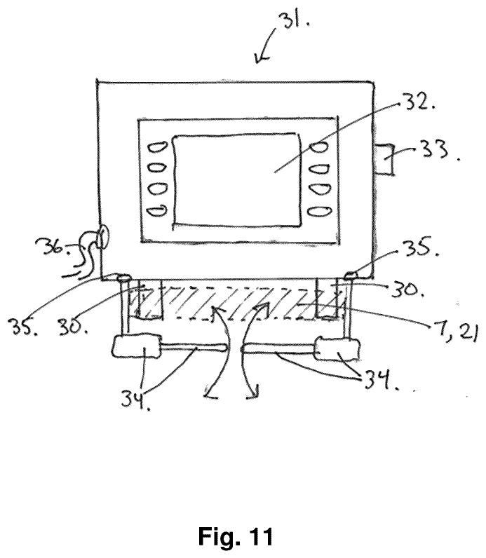

e.g. in reference to FIGS. 9 and 11, one embodiment of the present invention provides a handling machine (31), the handling machine comprising:
holding means (30),
one or more mixing paddles (34),
mixing paddle attachment means (35)
a control panel (32)
   wherein the speed and amount of physical force applied by the mixing paddles is adjustable utilizing the control panel, and/or the mixing paddles can be removed and re-attached utilizing the mixing paddle attachment means.

E.g. in reference to FIGS. 10 and 11, one embodiment of the present invention provides a handling machine (31), the handling machine comprising:
holding means (30),
a movable bar (37),
   wherein the movable bare is constructed for exerting physical force to the surface of the polymer bag and/or bags attached to the holding means (30) of the handling machine (31) the physical force applied by the movable bar gliding vertically op-and-down, and thereby squishing the surface of the polymer bag and/or bags attached to the machine.

In one embodiment of the present invention, the polymer layer, e.g. comprising the protrusions or buttons, for closing apertures (9) may be removed before applying physical force by the movable bar and thereby squishing the surface of the polymer bag and/or bags attached to the machine. In another embodiment of the present invention, the third bag (50) comprising the protrusions or buttons for closing apertures (9) may be removed before applying physical force by the movable bar and thereby squishing the surface of the polymer bag and/or bags attached to the machine. In yet another embodiment of the present invention, the separate polymer sheet (11), e.g. comprising the protrusions or buttons, for closing apertures (9) may be removed before applying physical force by the movable bar and thereby squishing the surface of the polymer bag and/or bags attached to the machine.

Yet an embodiment of the present invention, the separate single/polymer sheet, welded and or glued to the area of the apertures to closing or sealing the apertures, may be removed before applying physical force by the movable bar and thereby squishing the surface of the polymer bag and/or bags attached to the machine.

E.g. in reference to FIGS. 9 and 11, one embodiment of the present invention provides a handling machine (31), the handling machine comprising:
holding means (30),
a movable bar (37),
movable bar attachment means (38)
a control panel (32)
   wherein the speed and direction of the movement of the movable bar is adjustable utilizing the control panel, and/or the movable bar can be removed and re-attached utilizing the movable bar attachment means.

E.g. in reference to FIGS. 8, 9, 10 and 11, one embodiment of the present invention, provides a handling machine (31), the handling machine comprising:
holding means (30)
vacuum means (36)
   wherein the vacuum means (36) are constructed to be attached to the vacuum inlet (19) of the second bag attached to the holding means (30) of the handling machine (31).

Vacuum may commonly be utilized on polymer bags, e.g. for packaging different kind of both consumable such as food or medicine or for other purposes. A common vacuum means for applying vacuum on a polymer bag may be a vacuum pipe or a vacuum tube, that can be attached to the polymer bag by different known means, e.g. screwing and/or clamping and/or clicking to the handling machine.

One embodiment of the present invention, provides a handling machine (31), the handling machine comprising:
holding means (30)
dispensing means
   wherein the dispensing means may be constructed to be attached to the dosing outlet (18) of the second bag attached to the holding means (30) of the handling machine (31).

The dispensing means of the present invention are different dispensing means known in the art. One embodiment of the present invention provides a handling machine comprising dispensing means for constructing a fluid connection between the polymer second bag and flexible dosing means such as a dosing pipe; a dosing disc or dosing arm of a dosing liquid capsule filling machine, as provide by one of the embodiments of the present invention.

One embodiment of the present invention provides a handling machine (31) comprising:
holding means (30) for attaching at least one polymer bag according to the invention, to the holding machine;
one or more removable/re-attachable mixing paddles (34) for grinding, mixing and filtering the biological material comprised within the at least one polymer bag;
vacuum means (36) for applying vacuum to the at least one polymer bag according to the kit of the present invention;
removable/re-attachable movable bar (37) for exerting physical force to the at least one polymer bag according to the present invention;
   wherein the movable bar (37) squishes the biological material down towards the bottom wall of the polymer bag, in a downwards movement.

One embodiment of the present invention provides a handling machine comprising:
holding means for attaching one or more polymer bag of the kit of the present invention to the holding machine;
one or more mixing paddles for pushing, mixing and filtering the biological material comprised within the one or more polymer bags of the kit of the present invention;
dispensing means for attaching to the dosing outlet on the bottom wall on the one or more polymer bag of the kit of the present invention;
   and a movable bar for exerting physical force to the one or more polymer bag of the present invention, squishing the biological material down towards the bottom wall of the polymer bag in a downwards movement.

One embodiment of the present invention provides a capsule filling machine (such as a liquid capsule filling machine), comprising flexible dosing means, constructed for dosing a specific amount of filtered biological material into the bottom half of a capsule and closing the capsule by pressing an upper half capsule over the bottom comprising the filtered biological material.

The biological material dosed into the capsules may be the filtered biological material. Preferably, the filtered biological material according to the present invention may constitute the supernatant obtained from the biological material, in particular the faecal material. Preferably, the supernatant may be obtained from squishing the biological material from the first bag into the second bag.

An embodiment of the present invention provides a capsule filling machine that may be both easily cleaned and cooled, constructed for producing frozen capsules that are easy to handle, being more solid and durable than softer non frozen capsules produced by the capsule machines as known in the art.

Another embodiment of the present invention relates to a method of preparing a faecal capsule implant, the method comprising the steps of:
providing a faecal composition, preferably by the invention according to the present invention; and
filling the faecal composition into at least a first coating, providing a first coated faecal composition (a faecal capsule implant/a one layered faecal capsule implant).

In an embodiment of the present invention the faecal composition may be a liquid faecal composition.

The faecal composition may comprise at least one sample of stool from a healthy donor. Preferably the sample of stool may be placed in a sterile collection device.

In an embodiment of the present invention the sample of stool may be mixed with at least one diluent, preferably, the diluent comprising a cryoprotectant. Addition of cryoprotectant may preferably result in a final concentration of cryoprotectant in the faecal composition below 8% (v/v) cryoprotectant.

The mixture comprising the sample of stool and the diluent may be filtered providing a faecal composition comprising a stool fraction and a diluent, wherein the diluent comprises a cryoprotectant resulting in a final concentration of cryoprotectant in the faecal composition in the range of 1-15 % (v/v) cryoprotectant, such as in the range of 3-12% (v/v), e.g. in the range of 5-10% (v/v), such as in the range of 6-8% (v/v).

In an embodiment of the present invention, then after the faecal composition has been filled into at least a first coating, providing a first coated faecal composition, the first coated faecal composition may be enclosed by a second coating providing a capsule two layered capsule comprising the faecal composition (a faecal capsule implant/a two layered faecal capsule implant).

Preferably, the faecal capsule implant (the one layered faecal capsule implant and/or the two layered faecal capsule implant) may comprise an enteric delivery hard capsule, preferably, comprise only one enteric delivery hard capsule. Preferably, the enteric delivery hard capsule may comprise HPMC and/or hydroxypropyl methyl cellulose acetate succinate (HPMC-AS).

Preferably, the polymers capsule coating may comprise enteric delivery with low PH protection for oral dosage with acid resistance up to PH 6.0-7.

An embodiment of the present invention relates to a method for providing a capsule comprising a faecal composition, the method comprises the steps of:
(i) obtaining at least one sample of stool from a healthy donor;
(ii) placing said sample obtained in (i) in a sterile collection device;
(iii) mixing the sample obtained in (ii) with at least one diluent, preferably, the diluent comprises a cryoprotectant;
(iv) filtering the mixture obtained in (iii), providing the faecal composition comprising a stool fraction and the diluent, preferably, the diluent comprises a cryoprotectant resulting in a final concentration of cryoprotectant in the faecal composition with 1-15 % (v/v) cryoprotectant;
(v) filling the faecal composition into a first coating, providing a first coated faecal composition; and
(vi) optionally, enclosing the first coated faecal composition with a second coating providing the capsule comprising a faecal composition.

Wherein the first coating or the second coating comprises an enteric delivery hard capsule.

Preferably, the enteric delivery hard capsule comprises HPMC and/or hydroxypropyl methyl cellulose acetate succinate (HPMC-AS) polymers capsule coating with enteric delivery, preferably, with a low pH protection for oral dosage, preferably with an acid resistance up to PH 6.0-7.0.

In an embodiment the capsule of the present invention may comprise a hard capsule, preferably a pH sensitive hard capsule. Preferably, the capsule of the present invention may comprise a single coating comprising a pH sensitive hard capsule.

A further embodiment of the present invention relates to a faecal capsule implant obtainable by the present invention.

The process for of preparing a faecal capsule implant may preferably be performed under cold temperatures. Preferably, the cold temperatures relates to a temperature below 20° C., such as below 15° C., e.g. below 10° C., such as below 5° C., e.g. below 0° C., such as below 5° C., e.g. below −10° C., such as below −15° C., e.g. below −20° C., such as below −30° C., e.g. below −40° C., such as below −50° C., e.g. below −60° C., such as below −70° C., e.g. blow −80° C.

Preferably, the second bag may be used directly in the capsule filing machine. During preparation the second bag may be transferred directly from the handling machine to the capsule filing machine.

In an embodiment of the present invention the empty capsule filling/dosing disc may be provided with cooling.

Preferably, the cooling provided to the empty capsule filling/dosing disc may ensure a temperature of the empty capsule filling/ dosing disc below 20° C., such as below 15° C., e.g. below 10° C., such as below 8° C., e.g. below 6° C., such as below 4° C., e.g. below 2° C., such as below 0° C., e.g. below −2° C., such as below −4° C., e.g. below −6° C., such as below −8° C., e.g. below −10° C., such as below −15° C., e.g. below −20° C., such as below −30° C., e.g. below −40° C., such as below −50° C., e.g. below −60° C., such as below −70° C., e.g. below −80° C.

When the capsules are closed in the capsule filling machine the closed capsules containing the final faecal composition may be sealed or not sealed and transported to cold 6to −80 ° C., preferably about −20° C. container for further process.

The capsules provided according to the present invention may also provide doubled (2 layered) capsulated products. These capsules may be provided by doubled (2 layered) capsulated capsule filling machine, by adding additional capsule filling instrument/rotary to handle the second empty capsule code after the first capsulation with acid resistance capsules.

Or after first code with acid resistant capsule, when the finished capsules are transported to the −20 degrees container, the container will be filled in a plastic or metal container at the product/biological medicine station, where the acid resistance capsule may be capsulated with the second code of capsules in the liquid capsule filling machine.

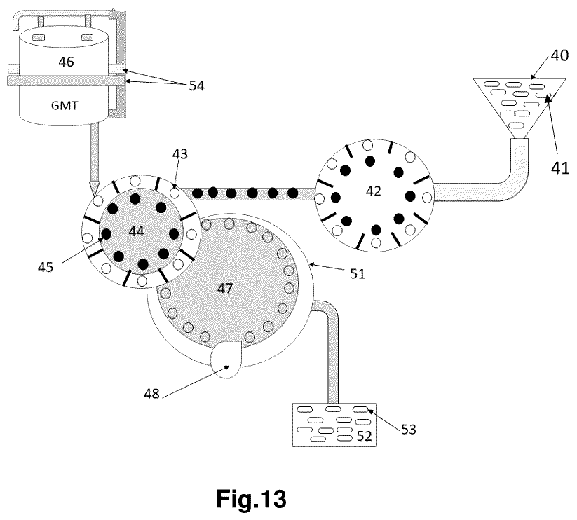

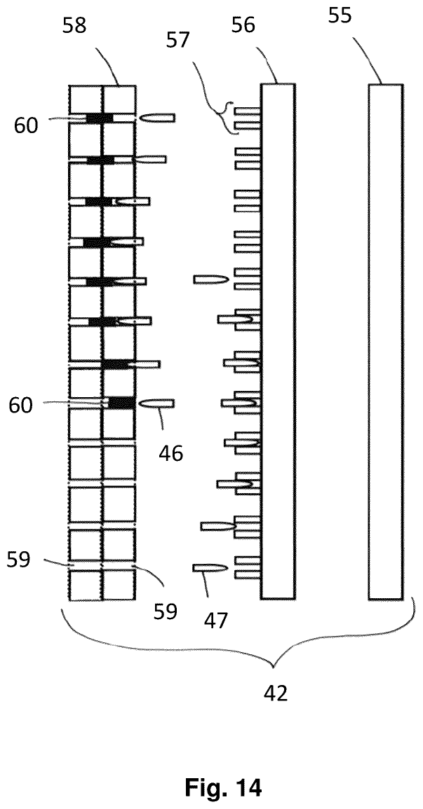

E.g. in reference to FIG. 13 a schematic process of liquid capsule filling machine (49) is shown. With reference to FIG. 13, the liquid capsule filling machine (49) comprises a empty capsule (41) dosage container (40) comprising a material suitable for easy cleaning, such as metal, in particular stainless or one time used hard plastic. The closed empty capsule is guided to disc (42) flow through the capsule hopper and into a capsule holder (not shown in figures) (42), where a fork grips the empty capsule and transfer it into the upper mould (not shown in figures) and lower mould (not shown in figures). A vacuum is applied (not shown in figures) for separating the capsule into upper and bottom capsule half's, resting in upper and lower moulds (not shown in figures) respectively. Separated empty capsules guided to empty capsule filling/dosing disc (44). In the empty capsule filling/dosing disc (44) the lower mould holding the empty capsule bottom (43) is filled with a filtered biological material (46), like FMT, e.g. using a polymer bag shown in FIG. 5 or FIG. 8 or a hard plastic dosing into the empty capsule bottom half, using sensor and or vacuum. The filtered biological material, FMT, may be dosed into the empty capsule bottom half by dosing means (54) that may squeeze the filtered biological material, FMT, out of the soft bag. The upper mould/upper half of the empty capsules (45) will be placed on top of the lower mould holding the empty capsule bottom (43) in capsule filling/dosing disc (44). Further in reference to FIG. 13, the split ejector pin (not shown in figures) closes the filled capsule comprising the two capsule parts and the dose of filtered biological materiel/FMT, by pressing the capsule button half up and into the capsule upper half that is kept in place by locking plate (not shown in figures). Then the closed filled capsule guided to disc (47), where the FMT comprising capsule sealed using heat (48) or not sealed using heat and the FMT filled capsuled guided to the cooled container (52).

One embodiment of the present invention provides a capsule filling machine, preferably a liquid capsule filling machine, comprising a capsule holder (42), said capsule holder (42) comprising a locking disc (55); an upper-mould (56); capsule locking means (57)and a lower mould (58) and wherein the capsule holder (42) of the capsule machine comprises material suitable for easy cooling and easy cleaning, such as metal, in particular stainless steel.

The capsule holder (42) comprising the upper-mould (56) further comprises capsule-holders (57) comprising a material suitable for easy cooling and easy cleaning, such as metal, particular stainless steel, constructed for holding the upper half of a capsule (47); and the lower mould (58) comprising a material suitable for easy cooling and easy cleaning, such as metal, in particular stainless steel, the lower mould further comprising holes (59) for holding the bottom half of a capsule (46) and split-ejector pins (60) constructed for pushing the bottom half of a capsule (46) op for closing the capsule.

One embodiment of the present invention provides a capsule filling machine comprising a capsule handling part, said capsule handling part comprising suitable metal, such as stainless steel and a suitable cooler, such as air-cooler, wherein the capsule handling part and the cooler are constructed for easy cooling of the capsule handling part of the capsule machine and production of filled capsules that are frozen and therefore easy to handle and with less odour issues.

During the process of preparing a faecal capsule implant, in particular a faecal capsule implant, from (and including) the empty capsule filling/dosing disc to the cooled container, the temperature may be controlled to ensure a temperature of the faecal composition below 20° C., such as below 15° C., e.g. below 10° C., such as below 8° C., e.g. below 6° C., such as below 4° C., e.g. below 2° C., such as below 0° C., e.g. below −2° C., such as below −4° C., e.g. below −6° C., such as below −8° C., e.g. below −10° C., such as below −15° C., e.g. below −20° C., such as below −30° C., e.g. below −40° C., such as below −50° C., e.g. below −60° C., such as below −70° C., e.g. below −80° C., such as in the range of −80-20° C., e.g. in the range of −60-15° C., such as in the range of −40-12° C., e.g. in the range of −30-9° C., such as in the range of −20-0° C., e.g. in the range of −15-10° C.

In an embodiment of the present invention the filtered biological material, e.g. the faecal composition, may during storage, and before being supplied to the capsule filling machine, be kept at a temperature below 20° C., such as below 15° C., e.g. below 10° C., such as below 8° C., e.g. below 6° C., such as below 4° C., e.g. below 2° C., such as below 0° C., e.g. below −2° C., such as below −4° C., e.g. below −6° C., such as below −8° C., e.g. below −10° C., such as below −15° C., e.g. below −20° C., such as below −30° C., e.g. below −40° C., such as below −50° C., e.g. below −60° C., such as below −70° C., e.g. blow-80° C., such as in the range of −80-20° C., e.g. in the range of −60-15° C., such as in the range of −40-12° C., e.g. in the range of −30-9° C., such as in the range of −20-0° C., e.g. in the range of −15-10° C.

One embodiment of the present invention provides a capsule machine comprising capsule handling part comprising metal, particular stainless steel and a cooler, particular air-cooler, constructed for easy cooling of the capsule machine to around −20° C., such as between −10° C. and −30° C., such as between −15° C. and −25° C.

One embodiment of the present invention provides a capsule filling machine, in particular a liquid capsule filling machine, (FIG. 13) for flexible dosing of filtered biological material into capsules (cold or not cold capsules), said capsules may previously have been cooled down, and then the closing, freezing and disposing of said filled capsules, said capsule machine comprising:
flexible dosing means connected to the dispensing means of the handling machine;
a metal locking plate (41), preferably comprising stainless steel, for providing pressure for closing the capsules after filling;
a metal upper-mould (45) preferably comprising stainless steel, said upper-mould further comprising multiple capsule holders (44) for holding the upper part of a capsule (44);
a metal lower mould (43) preferably comprising stainless steel, said lower mould further comprising multiple holes (44) for holding capsule bottoms (43) and spit-ejector pins.

One embodiment of the present invention relates to a method of mixing and/or filtering and/or dispensing of a biological material utilizing the kit and the holding machine of the invention, wherein the kit comprises at least one least one polymer bag.

Another embodiment of the present invention relates to a method of mixing, filtering and dispensing of a biological material in the form of a frozen or not frozen empty capsules, utilizing the kit provided by the invention, the holding machine of the invention and a dosing machine comprising stainless steel and wherein the kit comprises at least one least one polymer bag.

In an embodiment of the present invention the capsule filing machine may be an automatic or a semi-automatic capsule filling machine.

Preferably the capsule filing machine, or parts hereof may be cooled, e.g. using dry ice, liquid CO₂ or liquid nitrogen or the like.

Filling the empty capsules may be performed manually or automatically using a pump to dosage the desired amount of product into the capsules.

One embodiment of the present invention relates to a method of mixing and/or filtering and/or dispensing of a biological material, utilizing the kit of the present invention comprising at least one polymer bag (1 and/or 21) and the handling machine (31) of the invention, comprising the steps of:
fastening a kit comprising biological material contained within the main compartment (2 or 23) of at least one polymer bag to the holding means (30) of the handling machine,
applying one or more mixing paddles (34) to the attached kit,
removing the mixing paddles, utilizing the mixing paddle attachment means (35)
attach the movable bar (37) to the handling machine, by utilizing the movable bar attachment means (38)
applying the movable bar to the at least one polymer (1 and/or 21) bag attached to the handling machine,
   wherein the method optionally can comprise one or more of the steps of:
attaching vacuum means (36) to the vacuum inlet (19) of the second-bag (21) thereby applying vacuum to the polymer bag, and/or attaching flexible dosing means to the dosing outlet (18) of the second-bag (21), and/or
dispensing filtered biological material thorough the dosing outlet (18) the second-bag (21) to a liquid capsule filling machine (FIG. 13) comprising a metal part for handling the capsules, wherein the metal part of capsule machine is air-cooled to around −20° C. or only container 53 is air-cooled.
placing bottom half of capsules (43) in holes (44) of the lower mould of the capsule machine (44);
placing upper half of capsules (45) in capsule holders (44) of the upper mould of the liquid capsule filling machine (FIG. 13);
dosing a filtered biological material into the bottom half capsules (43) utilizing the locking plate (44) to close the filled capsules by pressing the upper half capsules over the bottom half capsules (data not shown);
applying the split-ejector pins to push the filled, closed and frozen or not frozen capsules.

In reference to the drawings, one embodiment of the present invention relates to a method of mixing and/or filtering and/or dispensing of a biological material, utilizing the holding machine (31) of the invention and the kit of the present invention, wherein the kit comprises:

### the Second Bag (21)

the main compartment (22) of the second bag comprising the first bag (1)
the main compartment (2) of the first bag comprising biological material,
the apertures on at least one of the opposing flat sides (3) of the first bag are closed by separate polymer sheet (11) or third bag (50), the method comprising the steps of:
fastening the kit to the handling machine (31),
applying one or more mixing paddles (34) to the attached kit, and wherein the method optionally comprises one or more of the steps of:
removing the kit from the holding machine, pulling/separating and discharging the separate polymer sheet (11) or third-bag (50) from the first bag (1) and/or re-placing the polymer first bag (1) within the main compartment (22) of the second bag, and/or
attaching the kit comprising both the first (1) and second bag (21) to the holding machine, and/or
attaching vacuum (36) to the attached kit, and/or
applying one or more mixing paddles (34) to the attached kit, and/or removing the mixing paddles, utilizing the mixing paddle attachment means (35), and/or attach the movable bar (37) to the handling machine, by utilizing the movable bar attachment means (38), and/or
applying the movable bar to the attached kit, and/or
attaching flexible dosing means to the attached kit, and/or dispensing filtered biological material thorough the dosing outlet (18) the second bag (21) to a liquid capsule filling machine (FIG. 13) comprising stainless steel, wherein the liquid capsule filling machine is air-cooled to around 6 till −20 ° C.
placing bottom half of capsules (43) in holes of the lower mould (44) of the liquid capsule filling machine (FIG. 13);
placing upper half of capsules (45) in capsule holders (44) of the upper mould of the liquid capsule filling machine (44);
dosing a filtered biological material into the bottom half capsules (43)
utilizing the locking plate to close the filled capsules by pressing the upper half capsules (45) over the bottom half capsules (43);
applying the split-ejector pins to push the filled, closed and/or frozen capsules from the mould to sealing disc (47).

Preferably, the present invention relates to a faecal capsule implant obtainable by the present invention.

The faecal capsule implant or the gut microbiota capsule implant according to the present invention may be used as a medicament to eradicate antibiotic resistant bacteria in patient colonized with it.

In an embodiment of the present invention the faecal capsule implant may remain stable for at least 7 days at −20° C., such as for at least 14 days, e.g. for at least 21 days, such as for at least 30 days, e.g. for at least 45 days, such as for at least 60 days, e.g. for at least 75 days, such as for at least 90 days, e.g. for at least 105 days, such as for at least 120 days, e.g. for at least 135 days, such as for at least 150 days.

In a further embodiment of the present invention the faecal capsule implant may remain stable for at least 30 days at −80° C., such as for at least 60 days, e.g. for at least 100 days, such as for at least 200 days, e.g. for at least 300 days, such as for at least 400 days, e.g. for at least 500 days, such as for at least 600 days, e.g. for at least 700 days. Preferably, the term “remain stable”, may relate to the preservation of viability of the microorganisms present in the faecal capsule implant. In an embodiment of the present invention the faecal capsule implant may remain stable resulting in at least 50% of the microorganisms in the faecal capsule implant be viable, such as at least 55%, e.g. at least 60%, such as at least 65%, e.g. at least 70%, such as at least 75%, e.g. at least 80%, such as at least 85%, e.g. at least 90%, such as at least 95%, e.g. at least 98%.

It should be noted that features described in the context of one of the embodiments or one or the aspects of the present invention also apply to the other embodiments and aspects of the invention.

1-17. (canceled)

18. A capsule filling machine for flexible dosing of filtered biological material into capsules, closing, freezing and disposing of said filled capsules, said capsule filling machine comprising:
flexible dosing means connected to the dispensing means of the handling machine
means for holding the polymer bag for collecting, storing, mixing and/or filtering a biological material, the at least one polymer bag comprising a main compartment enclosed by:
two opposing flat surfaces comprising polymer film,
two lateral side walls,
a bottom wall,
a top wall,
wherein at least one of the two opposing flat surfaces of at least one of the polymer bags of the kit, comprises multiple apertures;, containing the filtered biological material;
a metal empty capsule separating disc, preferably comprising stainless steel;
a metal empty capsule separating disc, where lower mould, preferably comprising stainless steel, said lower mould further comprising multiple holes and spit-ejector pins.
a metal empty capsule filling/dosing disc, comprising multiple holes and spit-ejector pins.

19. The capsule filling machine according to claim 18, wherein the empty capsule filling/dosing disc is provided with cooling.

20. The capsule filling machine according to claim 19, wherein the metal empty capsule separating disc, the metal empty capsule filling/dosing disc, and/or the metal empty capsule separating disc is cooled to −30° C. to 9° C.

21. The capsule filling machine according to claim 18, wherein the capsule filling machine is a liquid capsule filling machine.

22. The capsule filling machine according to claim 18, comprising a polymer first-bag and a polymer second bag, wherein the polymer first-bag is surrounded by the polymer second bag.

23. The capsule filling machine according to claim 18, wherein the multiple apertures are initially closed by a thin film, preferably a thin polymer film or wherein the multiple apertures are initially closed by a separate polymer sheet, such as a polymer sheet.

24. The capsule filling machine according to claim 18, wherein the multiple apertures have a diameter in the range of 0.1-3.5 mm, e.g. in the range of 0.3-3.0 mm, such as in the range of 0.5-2.5 mm, e.g. in the range of 0.7-2.0 mm, such as in the range of 1.0-1.75 mm, in the range of 1.2-1.5 mm.

25. The capsule filling machine according to claim 18, wherein one of the walls of the second bag comprises a closed vacuum inlet comprising vacuum attachment means and wherein the bottom wall of a second bag comprises a closed dosing outlet comprising dosing attachment means.

26. The capsule filling machine according to anyone of claim 18, wherein the first bag and/or a second bag and/or a separate polymer sheet comprise a solid material comprising an odour-barrier comprising polyvinylidene chloride (PVDC) and/or ethylene vinyl alcohol (EVOH) and/or polyvinyl alcohol (PVOH) and/or polyamide homo-, co-, and/or ter-polymers and wherein the outermost layer of the solid polymer material comprises flexible polyvinyl Chlorine (PVC) and/or and/ or oriented polypropylene (OPP) and/or Polyethylene (PE) terephthalate (PET) and/ or cast polypropylene (CPP).

27. A kit of a least one polymer bag for collecting, storing, mixing and/or filtering a biological material, the at least one polymer bag comprising a main compartment enclosed by:
two opposing flat surfaces comprising polymer film,
two lateral side walls,
a bottom wall,
a top wall,
wherein at least one of the two opposing flat surfaces of at least one of the polymer bags of the kit, comprises multiple apertures, wherein the multiple apertures are initially closed by a thin film, preferably a thin polymer film by a separate polymer sheet, such as a polymer sheet.

28. A kit of at least one polymer bag according to claim 27, wherein the multiple apertures have a diameter in the range of 0.1-3.5 mm, e.g. in the range of 0.3-3.0 mm, such as in the range of 0.5-2.5 mm, e.g. in the range of 0.7-2.0 mm, such as in the range of 1.0-1.75 mm, in the range of 1.2-1.5 mm.

29. A handling machine for mixing, filtering and dispensing of a biological material, comprising:
i. holding means provided for attaching one or more polymer bags of the kit according to claim 27, to the holding machine;
ii. at least one removable and re-attachable mixing paddle;
characterized in that the machine furthermore comprises a removable and re-attachable movable bar.

30. A method of mixing and/or filtering and/or dispensing of a biological material, utilizing the kit according to claim 27 and a handling machine for mixing, filtering and dispensing of a biological material, comprising the steps of:
fastening a kit comprising at least one polymer bag to the handling machine, the kit comprising biological material,
applying one or more mixing paddles to the kit, or part of the kit, attached to the handling machine, wherein the handling machine comprises:
i. holding means provided for attaching one or more polymer bags of the kit to the holding machine; and
ii. at least one removable and re-attachable mixing paddle;
characterized in that the holding machine furthermore comprises a removable and re-attachable movable bar.

31. A method of preparing a faecal capsule implant, the method comprising the steps of:
providing a faecal composition, preferably by a method according to claim 30; and
filling the faecal composition into at least a first coating, providing a first coated faecal composition (a faecal capsule implant/a one layered faecal capsule implant) using a capsule filling machine for flexible dosing of filtered biological material into capsules, closing, freezing and disposing of said filled capsules, said capsule filling machine comprising:
flexible dosing means connected to the dispensing means of the handling machine;
means for holding the polymer bag for collecting, storing, mixing and/or filtering a biological material, the at least one polymer bag comprising a main compartment enclosed by:
two opposing flat surfaces comprising polymer film,
two lateral side walls,
a bottom wall,
a top wall,
wherein at least one of the two opposing flat surfaces of at least one of the polymer bags of the kit, comprises multiple apertures containing the filtered biological material;
a metal empty capsule separating disc, preferably comprising stainless steel;
a metal empty capsule separating disc, where lower mould, preferably comprising stainless steel, said lower mould further comprising multiple holes and spit-ejector pins; and
a metal empty capsule filling/dosing disc, comprising multiple holes and spit-ejector pins.

32. A faecal capsule implant comprising a single acid sensitive hard capsule.
